(19) United States
(12) Patent Application Publication
(10) Pub. No.: US 2012/0064627 A1
(43) Pub. Date: Mar. 15, 2012

(54) APPARATUS AND METHOD FOR CULTURING STEM CELLS

(75) Inventors: Michelle Khine, Merced, CA (US); Chi-Shuo Chen, Merced, CA (US); Wei-chun Chin, Merced, CA (US)

(73) Assignee: The Regents of the University of California

(21) Appl. No.: 13/145,546

(22) PCT Filed: Jan. 25, 2010
(86) PCT No.: PCT/US10/21992

§ 371 Date: Nov. 29, 2011

Related U.S. Application Data
(60) Provisional application No. 61/147,424, filed on Jan. 26, 2009.

(51) Int. Cl. C12N 5/02; B29C 59/02; C12M 1/00

(52) U.S. Cl. 435/377; 435/383; 435/305.2; 264/219

(57) ABSTRACT

A method is provided to prepare a plurality of microwells suitable for the formation of embryoid bodies from embryonic stem cells. The method requires applying an image-forming material to a heat sensitive thermoplastic material in a designed pattern and heating the material under conditions that reduce the size of the receptive material by at least about 60% to create a mold. A polymer such as PDMS is then applied to the mold and removed to form the microwells. In an alternative aspect, the plurality of microwells on receptive material are prepared by etching a microwell designed pattern into a heat sensitive thermoplastic material support and then heating the material under conditions that reduce the size of the receptive material by at least about 60%.

### CROSS REFERENCE TO RELATED APPLICATIONS

This application claims the benefit under 35 U.S.C. §119(e) of U.S. Provisional Application Ser. No. 61/147,424, filed Jan. 26, 2009, the contents of which are incorporated by reference in its entirety.

### BACKGROUND OF THE INVENTION

Throughout this disclosure, various technical and patent publications are referenced to more fully describe the state of the art to which this invention pertains. These publications are incorporated by reference, in their entirety, into this application.

Three-dimensional spheroid culture systems (TDSCSs) are well known in the art. Researchers in tumor biology have used TDSCSs to study tumor cell biology, therapy resistance, cell-cell interactions, invasion, drug penetration, modeling, tumor markers, nutrient gradient, and tumor cell metabolism. Other reported uses include the study of numerous cell types such as mammary cells, hepatocytes, bone marrow cells and neural stem and progenitor cells. Paragraph [0003] of US Pat. Pub. 2007/0148767A1. The culturing of embryonic stems cells (ES) is particularly well suited to TDSCS because attachment of the stem cells to the culture surface may cause unwanted differentiation of the cells.

The ability to recapitulate embryogenesis in vitro is a potentially powerful tool. For example, studying the effect of genetic mutations on developmental processes or screening small molecule libraries which direct stem cell differentiation can be highly informative in many fields such as developmental biology, drug discovery, and tissue engineering (Mehta et al. Cell Biol. Int., 2008, 32:1412-1424; and Bratt-Leal et al. Biotechnol. Prog., 2009, 25:43-51). When grown in suspension, under differentiating conditions, embryonic stem cells (ESCs) form three-dimensional aggregates known as embryoid bodies (EBs) comprised of cells from the three primary germ layers, i.e. endoderm, mesoderm, and ectoderm. (Bratt-Leal et al. Biotechnol. Prog., 2009, 25:43-51; Zhang et al. Nat. Biotechnol., 2001, 19:1129-1133; Yirme et al. Stem Cells Dev., 2008, 171227-1241; Schuldiner et al. Brain Res., 2001, 913:201-205; Sachlos and D. T. Auguste, Biomaterials, 2008, 29:4471-4480; Reubinoff et al. Nat. Biotechnol., 2001, 19:1134-1140; Nishikawa et al. Development, 1998, 125:1747-1757; Kaufman et al. Proc. Natl. Acad. Sci. U.S.A., 2001, 98:10716-10721; Bigas et al. Blood, 1995, 85:3127-3133).

Prior art methods to produce EBs include the hanging drop technique (Banerjee and Bhonde (2006) Cytotechnology 51(1):1-5), low attachment method (U.S. Pat. No. 6,602,711), liquid bioreactors (Dang et al. (2004) Stem Cells 22(3):275-282), encapsulated liquid suspension culture (Pat. Publ. No. WO 03/004626), semisolid cellulose system (US Patent Publ. No. 2007/0148767A1) and semisolid culture by high viscosity medium and three-dimensional (3-D) culture (Stephen et al. (2002) Biotechnol. Bioeng. 78(4):442-453) and US Pat. Publ. No. 2005/0054100). Dan et al. in “Efficiency of Embryoid Body Formation and Hematopoietic Development from Embryonic Stem Cells in Different Culture System” (2002) Biotechnol. Bioen. 78:442-253, reviews and compares liquid suspension culture, methylcellulose culture, liquid attachment culture and hanging drop to differentiate stem cells to hematopoietic precursor cells.

Successful EB production requires a careful mix of cell and nutrient composition absent adherence of the cells to a surface. The above noted methods provide methods to grow EBs but the methods are not amenable to scale up. Paragraphs [0008] of US Patent Publ. No. 2005/0118711 A1. Modifications of these techniques for potential larger scale production of EBs are described in US Patent Publ. Nos. 2005/01187A1; 2006/286666A1; 2008/0026460A1 and 2008/0145925. However, these methods require costly manufacture of devices. In contrast, this method provides a simple, rapid and scalable culture method to load pre-defined cell numbers into microfabricated wells.

### SUMMARY OF THE INVENTION

Embryoid bodies (EB) are aggregates of embryonic stem cells. EB formation closely recapitulates early embryonic development with respect to lineage commitment. Because it is greatly affected by cell-cell and cell-substrate interactions, the ability to control the initial number of cells in the aggregates and to provide an appropriate substrate are important parameters for uniform EB formation. The most common way of creating these aggregates is the hanging drop method, a laborious approach of pipetting an arbitrary number of cells into well plates. The interactions between the stem cells forced into close proximity of one another promotes the generation of the EBs. Because the media in each of the wells has to be manually exchanged every day, this approach is manually intensive.

Moreover, because environmental parameters including cell-cell, cell-soluble factor interactions, pH, and oxygen availability can be functions of EB size, cell populations obtained from traditional hanging drops can vary dramatically even when cultured under identical conditions. Recent studies have indeed shown that the initial number of cells forming the aggregate can have significant effects on stem cell differentiation. This invention provides an apparatus and simple, rapid, and scalable culture method to load pre-defined numbers of cells into microfabricated wells and maintain them for embryoid body development. Finally, these cells are easily accessible for further analysis and experimentation. This method is amenable to any lab and requires no dedicated equipment.

In one aspect, this invention provides a method to prepare a plurality of wells in a thermoplastic material, by applying a first material to a second material in a designed pattern, wherein the second material is the thermoplastic material, and then heating the first and second material under conditions that reduce the length and width of the second material by at least 20% and increase the thickness by at least 120% of the area of the second material to which the first material is applied, thereby producing a mold. Thereafter, the mold is used to prepare the plurality of wells on a third material.

In another aspect, this invention provides a method to prepare a plurality of wells on a receptive material by etching a designed pattern into a thermoplastic material and then heating the material under conditions that reduce the length and width of the second material by at least 20% and increase the thickness of the second material by 120%, thereby preparing the plurality of wells, wherein the average diameter of the plurality of wells is from about 10 micrometers to about 2000 micrometers and wherein the average depth of the plurality of wells is at least 80% of the average diameter of the plurality of wells, thereby preparing a plurality of the wells.

Also provided by this invention is the apparatus produced by this method. The apparatus comprises a plurality of wells embedded or contained within a thermoplastic material. The wells are of a dimension and density such that they are useful to culture and/or propagate cells, such as eukaryotic stem cells. In one aspect, the apparatus for propagating cells comprises a plurality of wells on a polymer material, such as a thermoplastic polymer, wherein the average diameter of the plurality of wells is from about 10 micrometers to about 2000 micrometers and wherein the average capacity of the plurality of wells is from about 4 nanoliters to about 1 microliters, wherein the average depth of the plurality of wells is at least 80% of the average diameter of the plurality of wells.

In one aspect, the apparatus has intake and outtake ports that serve to remove and/or exchange liquid medium for the culturing and/or propagation of cells.

Further provided is a method for propagating and/or culturing cells such as stem cells by depositing into the wells of the apparatus of this invention a composition comprising at least one cell in an appropriate culturing and/or propagation medium. This can be accomplished by depositing the apparatus into a test tube having the cell composition and then centrifuging the test tube. The centrifugal force loads the cells into the wells. The apparatus having the cells deposited into the wells is then removed. After an appropriate amount of time and under appropriate conditions, the medium is removed and/or exchanged.

Also provided is a kit comprising a thermoplastic material and instructions for making a plurality of wells using the thermoplastic material, wherein the average diameter of the plurality of wells is from about 10 micrometers to about 2000 micrometers, wherein the average capacity of the plurality of wells is from about 4 nanoliters to about 1 microliter, wherein the average depth of the plurality of wells is at least 80% of the average diameter of the plurality of wells.

Yet further is provided a kit comprising a thermoplastic material, a polydimethylsiloxane prepolymer, and instructions for making a plurality of wells using the thermoplastic material and the polydimethylsiloxane prepolymer, wherein the average diameter of the plurality of wells is from about 10 micrometers to about 2000 micrometers, wherein the average capacity of the plurality of wells is from about 4 nanoliters to about 1 micrometer and wherein the average depth of the plurality of wells is at least 80% of the average diameter of the plurality of wells.

### BRIEF DESCRIPTION OF THE DRAWINGS

FIG. 1 shows Shrinky Dinks mold generation. The large image is an unshrunken Shrinky Dink Master with laser printed master pattern. The inset shows the master after being baked. In this image, the master shrunk to about 60% of the original size.

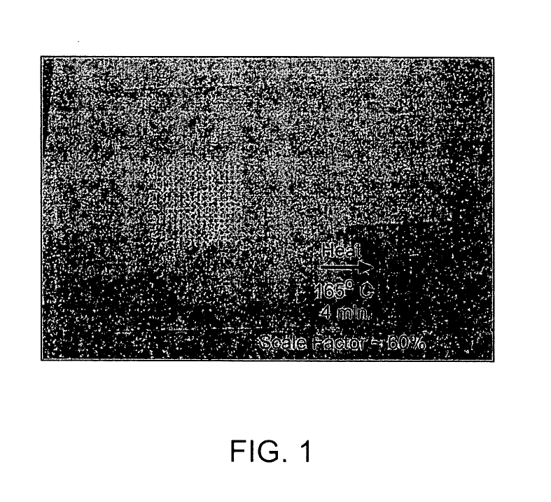

FIG. 2 shows the pattern of microwells on the PDMS made from the mold. The inset is a cross section of a PDMS well having a diameter of about 100 micrometers.

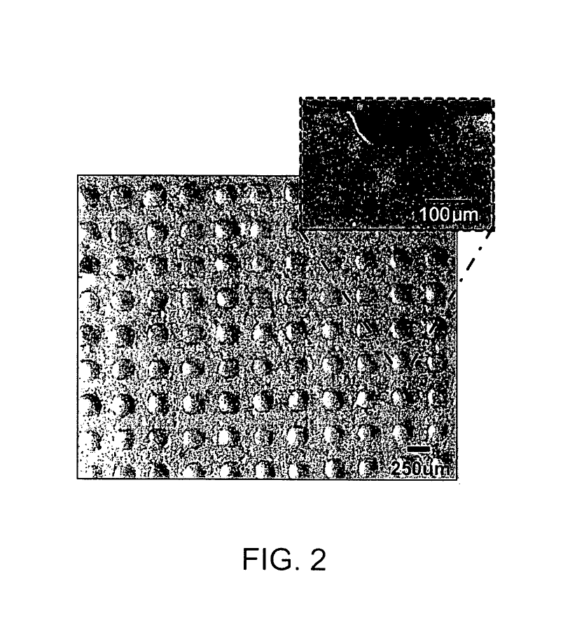

FIG. 3 is a graph showing the number of cells per well as a function of concentration of cells per ml of cell medium. The inset is a representative picture of cell loading.

FIG. 4A is picture showing an apparatus having input and output channels that feed the cells in the apparatus. FIG. 4B is a close-up of the channels feeding the cells.

### DETAILED DESCRIPTION OF THE INVENTION

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used herein, certain terms may have the following defined meanings

As used in the specification and claims, the singular form “a,” “an” and “the” include plural references unless the context clearly dictates otherwise. For example, the term “a microwell” includes a plurality of microwells.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps for preparing the microfluidic device. Embodiments defined by each of these transition terms are within the scope of this invention.

A “thermoplastic material” is intended to mean a plastic material which shrinks upon heating. In one aspect, the thermoplastic materials are those which shrink uniformly without distortion. The shrinking can be either bi-axially (isotropic) or uni-axial (anisotropic). Suitable thermoplastic materials for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC) and spectralon. A “Shrinky-Dink” is a commercial thermoplastic material which is marketed as a children's toy. As used herein, the terms “thermoplastic base” and “thermoplastic cover” refer to thermoplastic material having been subjected to both the etching process as well as heating process. The “thermoplastic base” would be located at the bottom or within the device, and the “thermoplastic cover” is the last layer of one or more layers of thermoplastic base.

A “well” is intended to mean a depression which is disposed within one or more levels of the microwell structure. The term “microwell” is generally defined as a substrate or material having a fluid depression with at least one internal cross-sectional dimension that is less than about 2000 micrometers and typically between about 0.1 micrometers and about 1500 micrometers which can be used in any number of biochemical or biological processes involving very small amounts of fluid. Such processes include, but are not limited to, containing and/or propagating cell compositions such as stem cells as described herein.

A “channel” is intended to mean a flow path which is disposed between the microwells. The term “microfluidic” is generally defined as a substrate or material having a passage through which a fluid, solid or gas can pass with at least one internal cross-sectional dimension that is less than about 500 micrometers and typically between about 0.1 micrometers and about 500 micrometers which can be used in any number of chemical processes involving very small amounts of material fluid. Such processes include, but are not limited to, electrophoresis (e.g., capillary electrophoresis or CE), chromatography (e.g., liquid chromatography), screening and diagnostics (using, e.g., hybridization or other binding means), and chemical and biochemical synthesis (e.g., DNA amplification as may be conducted using the polymerase chain reaction, or “PCR”) and analysis (e.g., through enzymatic digestion).

In addition to the above uses, the microfluidic channels disclosed herein can be patterned for “microfluidic mixing.” As used herein, the term “microfluidic mixing” is intended to mean the use of a receptive material having at least two inlet channels, wherein the inlet channels meet or intersect at an overlap region that may be in fluid communication with an outlet channel, such that fluids, such as solutions or other material, introduced from the inlet channels are mixed and may proceed into an outlet channel.

A “solution” is intended to refer to a substantially homogeneous mixture of a solute, such as a solid, liquid, or gaseous substance, with a solvent, which is typically a liquid. The solution can be either aqueous or non-aqueous. Examples of suitable solutes in solutions include fluorescent dyes, biological compounds, such as proteins, DNA and plasma, and soluble chemical compounds. Examples of suitable solids include beads, such as polystyrene beads, and powders, such as a metal powder. A “suspension” is intended to refer to a substantially heterogeneous fluid containing a solid, wherein the solid is dispersed throughout the liquid, but does not substantially dissolve. The solid particles in a suspension will typically settle as the particle size is large, compared to a colloid, where the particle size is small such that the suspension does not settle. Examples of suitable suspensions include biological suspensions such as whole blood, cell compositions, or other cell containing mixtures. It is contemplated that any solution, solid or suspension can be mixed using the mixers disclosed herein, provided that the solid has a particle size sufficiently small to move throughout the channels in the mixer.

A “composition” is intended to mean a combination of active agent, cell or population of cells and another compound or composition, inert (for example, a detectable agent or label) or active, such as a biocompatible scaffold.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype).

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

“Soft-lithography” is intended to refer to a technique commonly known in the art. Soft-lithography uses a patterning device, such as a stamp, a mold or mask, having a transfer surface comprising a well defined pattern in conjunction with a receptive or conformable material to receive the transferred pattern. Microsized and nanosized structures are formed by material processing involving conformal contact on a molecular scale between the substrate and the transfer surface of the patterning device.

The term “receptive material” is intended to refer to a material which is capable of receiving a transferred pattern. In certain embodiments, the receptive material is a conformable material such as those typically used in soft lithography comprise of elastomeric materials, such as polydimethylsiloxane (PDMS). The thermoplastic receptive material, or thermoplastic material, is also a receptive material as it can be etched, for example.

“Imprint lithography” is intended to refer to a technique commonly known in the art. “Imprint lithography” typically refers to a three-dimensional patterning method which utilizes a patterning device, such as a stamp, a mold or mask.

A “mold” is intended to mean an imprint lithographic mold.

A “patterning device” is intended to be broadly interpreted as referring to a device that can be used to convey a patterned cross-section, corresponding to a pattern that is to be created in a target portion of the substrate.

A “pattern” is intended to mean a mark or design.

“Bonded” is intended to mean a fabrication process that joins materials, usually metals or thermoplastics, by causing coalescence. This is often done by melting the materials to form a pool of molten material that cools to become a strong joint, with pressure sometimes used in conjunction with heat, or by itself, to produce the bond.

The term “isolated” means separated from constituents, cellular and otherwise, in which the cell, tissue, polynucleotide, peptide, polypeptide, protein, antibody or fragment(s) thereof, which are normally associated in nature. For example, in one aspect an isolated polynucleotide is separated from the 3′ and 5′ contiguous nucleotides with which it is normally associated in its native or natural environment, e.g., on the chromosome or from cellular constituents in which it is normally associated in nature. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, Wis. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4. A clone is a line of cells that is genetically identical to the originating cell; in this case, a stem cell.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of cardiomyocytes.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

As used herein, the “lineage” of a cell defines the heredity of the cell, i.e. its predecessors and progeny. The lineage of a cell places the cell within a hereditary scheme of development and differentiation.

A derivative of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells, such as, but not limited to, stem cell derived cardiomyocytes.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cell that give rise to the lining of the intestine, and cells that give rise to pancreogenic and splanchogenic cells.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi K. et al. (2007) Cell advance online publication 20 Nov. 2007; Takahashi K. & Yamanaka S. (2006) Cell 126: 663-76; Okita K. et al. (2007) Nature 448:260-262; Yu, J. et al. (2007) Science advance online publication 20 Nov. 2007; and Nakagawa, M. et al. (2007) Nat. Biotechnol. Advance online publication 30 Nov. 2007.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

“Embryoid bodies or EBs” are three-dimensional (3-D) aggregates of embryonic stem cells formed during culture that facilitate subsequent differentiation. When grown in suspension culture, ES cells form small aggregates of cells surrounded by an outer layer of visceral endoderm. Upon growth and differentiation, EBs develop into cystic embryoid bodies with fluid-filled cavities and an inner layer of ectoderm-like cells.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein, e.g. a calcium handling protein, a t-tubule protein or alternatively, a calcium pump protein. In another aspects, the substantially homogenous population have a decreased (e.g., less than about 95%, or alternatively less than about 90%, or alternatively less than about 80%, or alternatively less than about 75%, or alternatively less than about 70%, or alternatively less than about 65%, or alternatively less than about 60%, or alternatively less than about 55%, or alternatively less than about 50%) of the normal level of expression than the wild-type counterpart cell or tissue.

An “effective amount” is an amount sufficient to effect beneficial or desired results. An effective amount can be administered in one or more administrations, applications or dosages.

A “subject,” “individual” or “patient” is used interchangeably herein, and refers to a vertebrate, preferably a mammal, more preferably a human. Mammals include, but are not limited to, murines, rats, simians, bovines, canines, humans, farm animals, sport animals and pets. The cells useful in this invention can be from any appropriate subject, such as a mouse (murine) or human patient.

### Methods for Preparing and Using Microwells

Methods have been developed as lower-cost alternatives to photolithography, the ‘gold standard’ for microfabrication and microfluidic device creation. Duffy et al. first introduced ‘rapid prototyping of masters’ whereby they used printed transparencies to replace the expensive chrome masks traditionally utilized in photolithography (Duffy D., et al. (1998) Anal Chem. 70: 4974-4984). The authors demonstrated the advantages of using rapid prototyping for masks over conventional photolithography and micromachining Despite its convenience, the method still requires the use of expensive photoresist, high-resolution printing, and contact lithography. Tan et al. obviated this issue by direct printing; they photocopied designs onto transparencies to fabricate microfluidic channel molds that ranged in height from 8-14 micrometer, depending on the darkness setting of the photocopy machine (Tan A., et al. (2001) Lab Chip 1: 7-9). Liu et al. developed a one-step direct-printing technique for the design and fabrication of passive micromixers in microfluidic devices, with a maximum channel height of 11 micrometer (Liu A., et al. (2005) Lab Chip 5: 974-978). Such shallow channels are adequate for many microfluidic applications but not amenable for use with large mammalian cells (>10 micrometer in diameter) as well as other applications, such as flowing chemotactic gradients across adherent cells in a channel with minimal shearing (Lin F., et al. (2004) Biochem. And Biophys. Res. Commun. 319: 576-581).

While Lago et al. introduced a way to circumvent the height limitation of single-layer ink by printing up to four times using a thermal toner transfer method onto a glass substrate, the maximum height obtained with this approach was 25 micrometer (Lago C. L., et al. (2004) Electrophoresis 25: 3825-3831). Vullev et al. demonstrated a non-lithographic fabrication approach of microfluidic devices by printing positive-relief masters with a laser-jet printer for detecting bacterial spores; the height of the channels, which is likewise dependent on the height of the ink, is limited to between 5-9 micrometer (Vullev V., et al. (2006) J. Am. Chem. Soc. 128: 16062-16072). To achieve deep channels, McDonald et al. introduced the use of solid object printing (SOP) to make PDMS molds in thermoplastics (McDonald J. C., et al. (2002) Anal. Chem. 74: 1537-1545). However, despite their versatility, solid object printers are considerably costly ($50,000.)

Furthermore, the majority of these methods (as well as conventional photolithography) produce rectangular cross section channels. Pneumatic valves, first introduced by Quake et al., important for many microfluidic applications, require microfluidic channels to be rounded such that they can be completely sealed upon valve closure (Unger M. A., et al. (2000) Science 288(5463): 113-116). Achieving rounded microfluidic channels using typical photolithographic techniques, however, is complicated and requires an extra re-flow step of the photoresist at high temperatures. Most recently, Chao et al. demonstrated an elegant rapid prototyping approach, coined microscale plasma templating (μPLAT), using water molds. This technique enables the creation of rounded channels that are difficult to make with photolithography, but still requires micromachined masks and plasma activation (Chao S. H., et al. (2007) Lab Chip Technical Note 7: 641-643).

In one aspect, this invention is a method to prepare a plurality of wells by applying a first image-forming material such as ink, to a material wherein the second material is a thermoplastic material such as polystyrene in designed well type pattern and then heating the under conditions that reduce the length and width of the thermoplastic material by at least 20% and increase the thickness by at least 120%, or alternatively at least 130%, or alternatively, at least 140%, or alternatively at least 150%, of the area of the second thermoplastic material to which the first lithographic material is applied, thereby producing a mold. In one aspect, the second material is reduced by approximately 40-80, 50-70, about 60, in in-plane size. Thereafter, preparing the plurality of wells on a third material is prepared via a procedure such as molding using the mold.

In general, an “image-forming material” is one which is compressed upon heating, bonds to the plastic and is durable (can be used as a mold for multiple iterations). For example, “image-forming material” is, in one aspect, intended to mean a composition, typically a liquid, containing various pigments and/or dyes used for coloring a surface to produce an image or text such as ink and printer toner. In addition to an ink, the image forming material can be a metal, such as gold, titanium, silver, a protein, a colloid, a dielectric substance, a paste or any other suitable metal or combination thereof. Examples of suitable proteins include biotin, fibronectin and collagen. Examples of suitable colloids include pigmented ink, paints and other systems involving small particles of one substance suspended in another. Examples of suitable dielectric substances include metal oxides, such as aluminum oxide, titanium dioxide and silicon dioxide. Examples of suitable pastes include conductive pastes such as silver pastes.

The image forming material can be applied to the thermoplastic material by a variety of methods known to one skilled in the art, such as printing, sputtering and evaporating. The term “evaporating” is intended to mean thermal evaporation, which is a physical vapor deposition method to deposit a thin film of metal on the surface of a substrate. By heating a metal in a vacuum chamber to a hot enough temperature, the vapor pressure of the metal becomes significant and the metal evaporated. It recondenses on the target substrate. As used herein, the term “sputtering” is intended to mean a physical vapor deposition method where atoms in the target material are ejected into the gas phase by high-energy ions and then land on the substrate to create the thin film of metal. Such methods are well known in the art (Bowden et al. (1998) Nature (London) 393: 146-149; Bowden et al. (1999) Appl. Phys. Lett. 75: 2557-2559; Yoo et al. (2002) Adv. Mater. 14: 1383-1387; Huck et al. (2000) Langmuir 16: 3497-3501; Watanabe et al. (2004) J. Polym. Sci. Part B: Polym. Phys. 42: 2460-2466; Volynskii et al. (2000) J. Mater. Sci. 35: 547-554; Stafford et al. (2004) Nature Mater. 3: 545-550; Watanabe et al. (2005) J. Polym. Sci. Part B: Polym. Phys. 43: 1532-1537; Lacour, et al. (2003) Appl. Phys. Lett. 82: 2404-2406.)

In addition, the image forming material can be applied to the thermoplastic material using “pattern transfer”. The term “pattern transfer” refers to the process of contacting an image-forming device, such as a mold or stamp, containing the desired pattern with an image-forming material to the thermoplastic material. After releasing the mold, the pattern is transferred to the thermoplastic material. In general, high aspect ratio pattern and sub-nanometer patterns have been demonstrated. Such methods are well known in the art (Sakurai, et al., U.S. Pat. No. 7,412,926; Peterman, et al., U.S. Pat. No. 7,382,449; Nakamura, et al., U.S. Pat. No. 7,362,524; Tamada, U.S. Pat. No. 6,869,735).

Another method for applying the image forming material includes, for example “micro-contact printing”. The term “micro-contact printing” refers to the use of the relief patterns on a PDMS stamp to form patterns of self-assembled monolayers (SAMs) of an image-forming material on the surface of a thermoplastic material through conformal contact. Micro-contact printing differs from other printing methods, like inkjet printing or 3D printing, in the use of self-assembly (especially, the use of SAMs) to form micro patterns and microstructures of various image-forming materials. Such methods are well known in the art (Cracauer, et al., U.S. Pat. No. 6,981,445; Fujihira, et al., U.S. Pat. No. 6,868,786; Hall, et al., U.S. Pat. No. 6,792,856; Maracas, et al., U.S. Pat. No. 5,937,758).

Suitable first image-forming material includes without limitation an ink, metal, protein, biodegradable material, fluorescent dye, battery material, polymer, or conductive polymer. The first material can be applied to the thermoplastic material by sputtering, evaporating, printing, depositing, or stamping. Applicants have successfully used commercially available inkjet and laser jet printers to transfer the first material to the second. The transfer of the first material may be performed in one or more steps prior to heating the second material to reduce its size.

The thickness of the image-forming material, such as ink or toner, onto the heat sensitive thermoplastic receptive material dictates the depth of the microfluidic wells channels on the receptive material. Therefore, using the methods described herein, one can predictably and reproducibly fabricate microwells having a known depth.

In certain embodiments, the image-forming material is applied to the heat sensitive thermoplastic receptive material by one or more method comprising sputter coating, evaporation, chemical vapor deposition, pattern transfer, micro-contact printing or printing. In some embodiments, it is applied by printing. The printing can be done using any suitable printer, such as a laser or ink jet printer or computer-controlled plotter, directly onto the thermoplastic material.

In an alternative embodiment, the image forming material is a metal. Various metals can be used as an image forming material in the methods of the disclosed invention such as gold, titanium, silver, or any other suitable metal or combination thereof. In certain embodiments, the metal is deposited by a procedure such as, for example, comprising, consisting essentially or yet further, sputter coating, evaporation or chemical vapor deposition.

In one aspect of the disclosed invention, the second material, which is a thermoplastic material, is one which is heat sensitive and shrinks uniformly without distortion along one or two dimensions (length and width or X and Y axis). Suitable thermoplastic materials for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC) and spectralon. The materials can be pre-shrunk in one or two dimensions, such that upon application of the first material and heating, it only shrinks in the second dimension.

In one aspect, the above methods are performed under conditions wherein the second material, which is thermoplastic is reduced in size by heating or other method known in the art such that the length and/or width of the second material is reduced by at least 20% of its original size prior to heating and increase the height of the first material is increased by at least 3 times of the area of the second material to which the first material is applied. Alternative embodiments of the methods include, but are not limited to the application of heat to reduce the size of the receptive material by at least 30%, or alternatively, at least 40%, or alternatively, at least 50%, or alternatively, at least 60%, or alternatively, at least 70%, or alternatively, at least 75%, or alternatively, at least 80%, or alternatively, at least 85%, or alternatively, at least 90%, or alternatively, at least 95%, which in one aspect is measured in in-plane size. The height of the first material alternatively can be increased by at least 3.25 times, or alternatively at least 3.5 times, or alternatively at least 3.75 times, or alternatively at least 4.0 times, or alternatively 4.25 times, or alternatively at least 4.5 times, the original height of the first material.

To create the mold from the reduced second material, which is thermoplastic, the third material is prepared by a process comprising, or alternatively consisting essentially of, or yet further consists of, lithography such as soft lithography or imprint lithography from the second material. Suitable third materials for use in this invention include, but are not limited to a polymer such as polydimethylsiloxane.

After the microwell plate has been prepared, the wells may be coated with a material or materials that can facilitate the growth and/or differentiation of the cells. Such materials include, but are not limited to a growth factor selected from the group consisting of fibronectin, polylysine, gelatin (e.g., 0.1%, Sigma-Aldrich) or an extracellular matrix protein.

Also provided by this invention is a method to prepare a plurality of wells on a receptive material, by etching a designed pattern of wells into a thermoplastic material and then heating the material under conditions that reduce the length and width of the receptive material by at least 20% and increases the thickness of the receptive material by at least 120%, thereby preparing the plurality of wells. Thereafter, the wells can be coated with a material or materials that can facilitate the growth and/or differentiation of the cells. Such materials include, but are not limited to a growth factor selected from the group consisting of fibronectin, polylysine, gelatin (e.g., 0.1%, Sigma-Aldrich) or an extracellular matrix protein.

The size, shape and capacity of the wells can be the same or different. The average diameter of the plurality of the wells ranges from about 10 micrometers to about 2000 micrometers, or alternatively from about 50 micrometers to about 1500 micrometers, or alternatively from about 50 micrometers to about 1500 micrometers, or alternatively from about 50 micrometers to about 100 micrometers, or alternatively from about 100 micrometers to about 2000 micrometers, or alternatively from about 100 micrometers to about 1500 micrometers, or alternatively from about 50 micrometers to about 800 micrometers, or alternatively from about 100 micrometers to about 800 micrometers, or alternatively from about 140 micrometers to 500 micrometers, or alternatively from about 100 to 250 micrometers, or alternatively from about 225 to about 325 micrometers, or alternatively from about 350 to 425 micrometers, or alternatively from about 400 to 500 micrometers, or alternatively from about 350 to 475 micrometers, or alternatively less than about 2000 micrometers, or alternatively less than about 2000 micrometers, or alternatively less than about 1500 micrometers, or alternatively less than about 1000 micrometer, or alternatively less than about 800 micrometers, or alternatively less than about 500 micrometers, or alternatively less than about 400 micrometers, or alternatively less than about 250 micrometers.

The wells can be in close proximity to each other, e.g., less than about 1000 micrometers from each other, or alternatively less than 900 micrometers from each other, or alternatively less than 800 micrometers from each other, or alternatively less than 700 micrometers from each other, or alternatively less than 600 micrometers from each other, or alternatively less than 500 micrometers from each other, or alternatively less than 400 micrometers from each other, or alternatively less than 900 micrometers from each other, or alternatively less than 300 micrometers from each other, or alternatively less than 250 micrometers from each other, or alternatively less than 100 micrometers from each other.

In addition, by modifying the amount of first material applied to the second material, and the heating or other method that reduces size (which in turn determines the shrinking) of the second material, one can prepare a variety of wells of different sizes and shapes, e.g., ovoid, spherical and/or square. The wells can vary in size and/or shape by multiple applications of the first material to the second and they can be the same or different from each other. For the purpose of illustration only, the first material can be applied in an amount that will produce spherical wells on the third material. The first material, in a different pattern, size or shape, can then be applied to the same second material. In this way, a plurality of wells of the same or differing size, dimension or capacity can be prepared. For example, the size of the wells can be the same or different and can have aspect ratios from about 4:1 to about 1:1, or alternatively, from about 3:1 to about 1:1 or alternatively from about 2:1 to about 1:1.

This invention also provides a support or apparatus for biological, chemical or other applications that comprises a polymer support such as polydimethylsiloxane having embedded within it a plurality of microwells of the same or different size, shape or dimension such as square shape, ovoid and/or spherical. For example, the size of the wells can be the same or different and can have aspect ratios from about 4:1 to about 1:1, or alternatively, from about 3:1 to about 1:1 or alternatively from about 2:1 to about 1:1.

The average diameter of the plurality of the wells ranges from about 10 micrometers to about 2000 micrometers, or alternatively from about 50 micrometers to about 1500 micrometers, or alternatively from about 50 micrometers to about 1500 micrometers, or alternatively from about 50 micrometers to about 100 micrometers, or alternatively from about 100 micrometers to about 2000 micrometers, or alternatively from about 100 micrometers to about 1500 micrometers, or alternatively from about 50 micrometers to about 800 micrometers, or alternatively from about 100 micrometers to about 800 micrometers, or alternatively from about 140 micrometers to 500 micrometers, or alternatively from about 100 to 250 micrometers, or alternatively from about 225 to about 325 micrometers, or alternatively from about 350 to 425 micrometers, or alternatively from about 400 to 500 micrometers, or alternatively from about 350 to 475 micrometers, or alternatively less than about 2000 micrometers, or alternatively less than about 2000 micrometers, or alternatively less than about 1500 micrometers, or alternatively less than about 1000 micrometer, or alternatively less than about 800 micrometers, or alternatively less than about 500 micrometers, or alternatively less than about 400 micrometers, or alternatively less than about 250 micrometers.

The wells may be in close proximity to each other, e.g., less than about 1000 micrometers from each other, or alternatively less than 900 micrometers from each other, or alternatively less than 800 micrometers from each other, or alternatively less than 700 micrometers from each other, or alternatively less than 600 micrometers from each other, or alternatively less than 500 micrometers from each other, or alternatively less than 400 micrometers from each other, or alternatively less than 900 micrometers from each other, or alternatively less than 300 micrometers from each other, or alternatively less than 250 micrometers from each other, or alternatively less than 100 micrometers from each other.

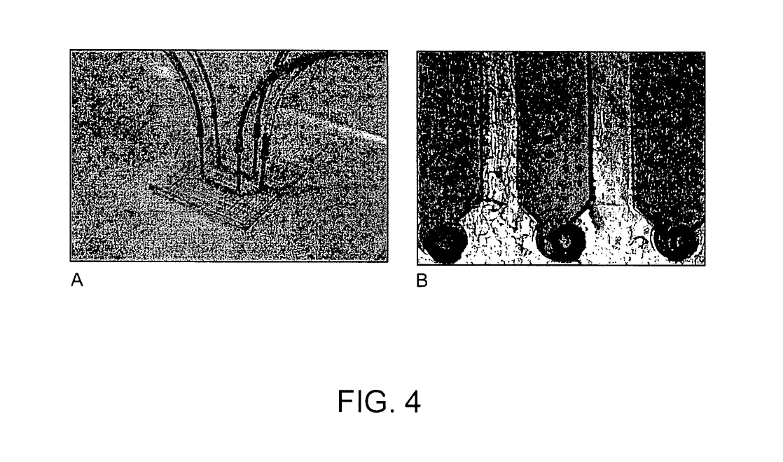

Microchannels can be created across the microwells and connected to a source of negative pressure as shown in FIG. 4. Media can be exchanged by attaching on port or tube to a source of cell culture media and another port, to a source of negative pressure such as a vacuum pump. By applying negative pressure, the spent culture media is removed from the microwell chip and new fresh media is pulled into the microwells with minimal disruption to the cells.

This invention also provides a method for growing or culturing cells such as stem cells, by applying a cell or cell in a suitable into at least one microwell of the apparatus described above. In one aspect, the invention provides materials and methods to grow EBs.

In one aspect the cell such as an embryonic stem cells, such as an animal or mammalian stem cell or cells are loaded by placing a microwell apparatus into a test tube having the cells suspended in an appropriate media. Methods for growing EBs are described in the literature such as Banerjee and Bhonde (2006) Cytotechnology 51(1):1-5, U.S. Pat. No. 6,602,711, Dang et al. (2004) Stem Cells 22(3):275-282), Pat. Publ. No. WO 03/004626, US Patent Publ. No. 2007/0148767A1, Stephen et al. (2002) Biotechnol. Bioeng. 78(4):442-453), US Pat. Publ. No. 2005/0054100 and Dan et al. in “Efficiency of Embryoid Body Formation and Hematopoietic Development from Embryonic Stem Cells in Different Culture System” (2002) Biotechnol. Bioen. 78:442-253,

The test tube containing the cells and apparatus are centrifuged at 760 rpm at 4° C. for approximately 5 minutes. After the centrifuge stops, the apparatus with the cells loaded into it is removed and placed into the appropriate environment for culturing and/or propagating the cells. When it is necessary to change the media or carrier, the media is carefully removed from the microwells without disturbing the cells. Alternatively, microfluidic channels, secured by vacuum pressure, are placed on top of the cell-filled microwells (see FIG. 4). This allows perfusion of cells in a combinatorial way with chemical stimuli. This allows one to add the microwell channels with the cells already loaded and easily retrieve the cells for further analysis off-chip.

### Kits

Also provided by this invention is a kit comprising, or alternatively consisting essentially, or yet further, consisting of, a) thermoplastic material and b) instructions for making a plurality of wells using the thermoplastic material as described above and incorporated herein by reference. For the purpose of illustration only, the average diameter of the plurality of wells is as described above, e.g., from about 10 micrometers to about 2000 micrometers, wherein the average capacity of the plurality of wells is as described above, e.g., from about 4 nanoliters to about 1 microliter, wherein the average depth of the plurality of wells is at least 80% of the average diameter of the plurality of wells. In another aspect, the kit further comprises a polymer, e.g., PDMS, or optionally, an image forming material.

In another aspect, the kit further comprises, or yet further consists essentially of, or yet further consists of, instructions for propagating cells in the plurality of wells. In one aspect, the instructions are for propagating eukaryotic stem cells, for example creating EBs in the plurality of wells.

This invention further provides a kit comprising, or alternatively consisting essentially of, or yet further consisting of, a) thermoplastic material, b) polydimethylsiloxane prepolymer, and c) instructions for making a plurality of wells using the thermoplastic material and the polydimethylsiloxane prepolymer. In another aspect, the kit further comprises, or alternatively consists essentially of, or yet further consists of, an image-forming material. The kit also provides instructions for making and using the apparatus described above and incorporated herein by reference. By way of example only, the instructions and materials are useful to prepare the microwell device wherein the average diameter of the plurality of wells is as described above, e.g., from about 10 micrometers to about 2000 micrometers, wherein the average capacity of the plurality of wells is as described above, e.g., from about 4 nanoliters to about 1 micrometer, wherein the average depth of the plurality of wells is as described above, e.g., at least 80% of the average diameter of the plurality of wells.

In another aspect, the kit further comprises, or yet further consists essentially of, or yet further consists of, instructions for propagating cells in the plurality of wells. In one aspect, the instructions are for propagating eukaryotic stem cells, for example creating EBs in the plurality of wells.

The following examples are intended to illustrate, but not limit the invention.

### Example 1

The microwells can be designed in AutoCad 2002 (AutoDesk, San Rafael, Calif.). Using a Hewlett-Packard LaserJet 2200D, designs are printed onto the polystyrene thermoplastic sheets (Shrinky Dinks, K & B Innovations, North Lake, Wis.) that resemble transparencies. These thermoplastic sheets are then fed through the printer several times for additional height and/or multi-dimensional wells. Printers can be set to either 600 dpi or 1200 dpi. Counter-intuitively, at 600 dpi, smoother feature edges were achieved, at the expense of height. The transparency setting was used for the printer. For multi-layered printing, alignment was ensured by adjusting the printer paper tray such that it tightly fit the thermoplastic sheet. Various printers have been tried, including a HP Color LaserJet 2600n and a Samsung ML-2510. The primary difference between the various printers was a slight variation in ink height.

The printed polystyrene sheet is placed in an oven for about 3-5 minutes at 163° Celsius. Both a standard toaster oven as well as a laboratory-grade oven can be used. Whereas slight warping can result from the toaster over, heating in the pre-heated lab oven resulted in more uniform heating. The devices were heated on a glass microscope slide for even more uniform and flat baking. It was found that the slides should not be pre-heated or they will melt the plastic.

The thermoplastic sheet naturally curls while shrinking to make the mold. Uniform heat on a flat surface will ensure that the thermoplastic sheet will re-flatten after complete shrinking A post-bake of 7 minutes in the oven after shrinkage greatly smoothes the ink features, and helps maintain ink adhesion. Devices have been molded over ten times with the same patterning device without any noticeable deterioration in the mold.

The PDMS is poured onto the mold as in typical soft lithography, and cured at 110° Celsius for 10 minutes. The cured PDMS device is then peeled off the mold and bonded using a hand-held corona discharger (Haubert K., et al. (2006) Lab Chip Technical Note 6: 1548-1549). The whole process from device design conception to working device can be completed within minutes.

To address the need to create deep and rounded microfluidic wells without expensive and dedicated tooling, a novel method of printing microfluidic channel networks onto commercially available thermoplastic “Shrinky-Dinks” in a standard laser-jet printer is disclosed herein. “Shrinky-Dinks” are a children's toy onto which one can draw a picture and subsequently shrink it to a small fraction of its original size. It was discovered that when features are printed onto this thermoplastic, after heating for 3-5 minutes at 163° Celsius, the printed features shrink isotropically in plane by approximately 63% from the original printed line width and length. There is an additional corresponding increase in height of the features by over 500%. Therefore, these shrunken features were subsequently used as a rigid mold for soft lithography (Xia Y., et al. (1998) Annu Rev. Mater. Sci. 28: 153-84). The thermoplastic mold is thus analogous to the commonly-used silicon wafer, which typically requires photolithographic patterning, for microfluidic applications. Like its silicon wafer counterpart, these plastic molds can be reused numerous times. Unlike the expensive setup and laborious processing required to make the silicon wafers, this approach only requires a laser-jet printer and a toaster oven, and can be completed within minutes. Moreover, multi-height designs within can be achieved the device, which typically requires a laborious and iterative process using standard lithographic approaches.

This invention presents a simple method to fabricate microfluidic wells of various size and dimension. The ability to create molds by printing at a larger scale and then shrinking down more than about 60% by leveraging the inherent property of thermoplastics is demonstrated.

The ability to rapidly, easily, and inexpensively create 2-D or 3-D plastic microfluidic chips will enable researchers of all academic fields, even with no engineering backgrounds, to design devices specific to their needs. In addition, elimination of the need for PDMS greatly increases the range of applications for which these microfluidic chips can be utilized. Combining simple and rapid fabrication, three dimensional complexity, and chemical compatibility will undoubtedly help usher microfluidics from the prototyping stage to its full potential of miniaturized system for addressing critical biomedical issues.

### Example 2

The method of this invention is illustrated by creating embryoid bodies using a red fluorescent mouse cell line (129S6B6-F1). A video demonstration of the process is available on the Journal of Visualized Experiments, available at the web site jove.com/index/details.stp?ID=692, first published on line on Mar. 9, 2008.

A desired pattern on shrinky-dink sheet was made using a good definition printer. The shrinky-dink was heated at 163° C. for about 10 minutes, or until fully shrunk and having acquired a regular shape. After shrinky-dink mold has cooled down, it was submerged it in an isopropanol bath until the complete surface is barely covered.

Some acetone was carefully sprayed over the mold and the container was shaken for a few times. More isopropanol was added to wash out acetone excess and this step was repeated a few times until the shrinky-mold looked clean. The mold was then immersed in distilled water for 10 minutes to wash off any remaining organic solvent. The mold was then air-cleaned and re-heated for about 5 minutes at 163° C. This compacts the ink and evaporates any remaining solvent.

A 10:1 PDMS/curing agent (Sylgard 184, Dow Corning) was prepared and agitated vigorously for few minutes. The shrinky-dink mold was placed in a small petri dish and the PDMS mixture until it reached about ½ cm over the mold surface. The dish was placed under a vacuum bell to eliminate all bubbles from PDMS mixture. The dish was then placed in an oven at 70° C., overnight.

The solid PDMS was cut from the mold and bound to a glass slide just by applying pressure. The first microwell-chip was discarded since it has ink residues incrusted between PDMS. The procedure was repeated to produce a second chip that was ink-free with a more defined shape.

The microwell-chip was cleaned using 70% ethanol solution and placed under UV light source for 10 minutes to sterilize it.

Cells were counted and diluted in culture media to the desired concentration (depending on how many initial cells in wells are desired). For example, to get approximately 10-15 cells per well (average=11, SD=5.4, loading rate=93%), a concentration of 8×10⁴ cells/ml. were used. For a concentration of 17×10⁴ cells/ml, one can reliably get between 25 and 35 cells per well (average=27.17857 SD =7.7, loading rate=100%).

The microwell chip is placed in a 50 ml centrifuge tube containing a solidified PDMS base. About 2 ml of the cell solution is added to the tube and the tube is centrifuged for 5 minutes at 760 rpm and 4° C.

The excess cell solution is pipetted from the tube and carefully washed with PBS 1× solution. The cell-loaded microwell was placed in a small petri dish after carefully taking the chip out of the centrifuge tube. The excess cell solution is carefully removed by washing with a 1×PBS solution.

To confirm the number of cells per well, the microwell chip was placed under an inverted microscope to count the cells and verify intended number of cells per well.

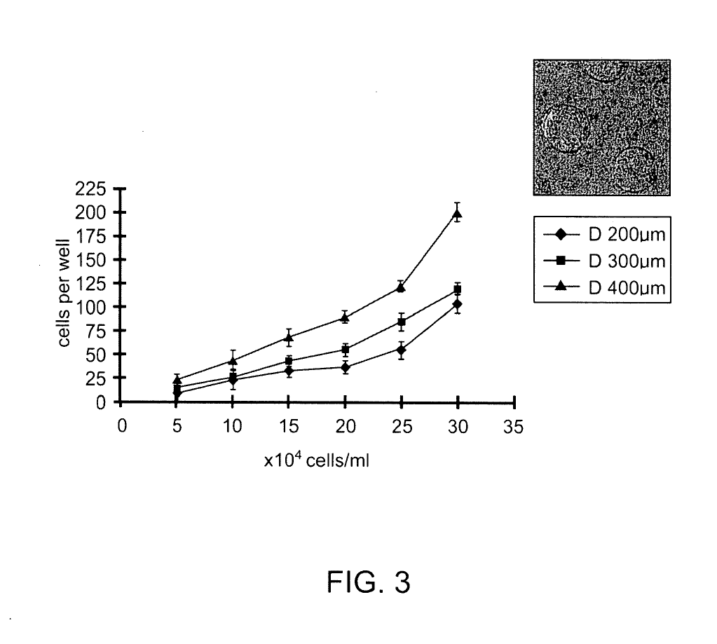

The cells were then incubated in the microwell under standard conditions. Cell medium can be changed from the side of the microwell avoiding disturbing the cells in the microwells. EBs easily grew in the wells (see FIG. 3).

While the present invention is exemplified and illustrated by the use of polystyrene sheets to fabricate channel structures and molds, it would be obvious to those of skill in the art that any thermoplastic receptive material that can be patterned to control the dimensions of the channel defining walls and thereby their size, can be used to fabricate the devices disclosed and claimed herein.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method to prepare an apparatus comprising a plurality of wells, this method comprising:
a) applying a first material to a second material in a designed pattern, wherein the second material is a thermoplastic material,
b) heating the first and second material under conditions that reduce the length and width of the second material by at least 20% and increase the thickness by at least 120% of the area of the second material to which the first material is applied, thereby producing a mold, and
c) preparing the plurality of wells on a third material via molding using the mold, thereby preparing the plurality of wells.

2. A method according to claim 1, wherein the second material of step b) is heated under conditions that reduce the length and width of the second material by at least 40% and increase the height by at least 3 times of the area of the second material to which the first material is applied.

3. A method according to claim 1, wherein the molding of step c) is lithography or imprint lithography.

4. (canceled)

5. A method according to claim 1, wherein the second material is a polymer or polystyrene.

6. (canceled)

7. A method according to claim 1, wherein the third material is a polymer or polydimethylsiloxane.

8. (canceled)

9. A method according to claim 1, wherein the first material is an image-forming material.

10. A method according to claim 1, wherein the first material is an ink, a metal, a protein, a biodegradable material, a fluorescent dye, a battery material, a polymer, or a conductive polymer.

11. A method according to claim 1, wherein the first material is applied to the thermoplastic material by sputtering, evaporating, printing, depositing, or stamping.

12. A method according to claim 1, further comprising repeating step a) one or more times prior to performing step b).

13. A method according to claim 1, wherein at least two of the plurality of wells differ from each other in a dimension and/or differ from each other in capacity.

14. (canceled)

15. A method according to claim 1, wherein the wells are one or more of: have an average capacity of from about 4 nanoliters to about 1 microliter; or having an average diameter of from about 10 micrometers to about 2000 micrometers; or have aspect ratios from about 1:4 to about 1:1.

16. A method according to claim 1, further comprising:
d) coating the wells with a factor selected from the group consisting of fibronectin, polylysine, gelatin or an extracellular matrix protein.

17. A method according to claim 1, wherein the dimension of the plurality of wells have aspect ratios of from about 4:1 to about 1:1.

18. An apparatus comprising a plurality of wells produced by the method of claim 1.

19. The apparatus of claim 18, wherein the apparatus further comprises at least one input channel and at least one output channel, and a channel connecting the input and output channel.

20. A method to propagate cells comprising growing the cells under conditions that favor the propagation of the cells in an apparatus of claim 18 or 19.

21. A method according to claim 20, wherein the conditions comprise substitution of the propagation medium.

22. A method according to claim 20, wherein the cells are selected from the group of embryonic stem cells (ESCs), somatic stem cells and induced pluripotent stem cells (iPSCs).

23. A method according to claim 21, wherein the propagation medium is selected to differentiate the cells along a pre-selected lineage or the formation of embryoid bodies.

24-30. (canceled)

31. A method to prepare an apparatus comprising a plurality of wells on a receptive material, the method comprising:
a) etching a designed pattern into a thermoplastic material; and
b) heating the material under conditions that reduce the length and width of the second material by at least 20% and increase the thickness of the second material by 120%, thereby preparing the plurality of wells, wherein the average diameter of the plurality of wells is from about 10 micrometers to about 2000 micrometers, wherein the average depth of the plurality of wells is at least 80% of the average diameter of the plurality of wells, thereby preparing a plurality of the wells on the receptive material.

32. A method according to claim 31, wherein the thermoplastic material is a polymer or polystyrene.

33. (canceled)

34. An apparatus comprising a plurality of wells produced by the method of claim 31.

35. An apparatus for propagating cells comprising a plurality of wells on a polymer material, wherein the average diameter of the plurality of wells is from about 10 micrometers to about 2000 micrometers, wherein the average capacity of the plurality of wells is from about 4 nanoliters to 1 microliter, wherein the average depth of the plurality of wells is at least 80% of the average diameter of the plurality of wells.

36-41. (canceled)

42. A kit comprising an apparatus of claim 35 and instructions for use of the apparatus.

43. A kit according to claim 42, further comprising instructions for propagating cells in the plurality of wells.

44-47. (canceled)
